(19) United States
(12) Patent Application Publication
(10) Pub. No.: US 2016/0135980 A1
(43) Pub. Date: May 19, 2016

(54) SINU Pads

(71) Applicant: Derrick D. Earls, Chicago, IL (US)

(72) Inventor: Derrick D. Earls, Chicago, IL (US)

(21) Appl. No.: 14/541,175

(22) Filed: Nov. 14, 2014

(51) Int. Cl. A61F 5/08; A61F 13/02

(52) U.S. Cl. CPC A61F 5/08; A61F 13/025

(57) ABSTRACT

A two layered device for opening nasal passages, the device comprising: a circular pad having an adhesive underlay surface, a smooth overlay surface, and a center soft plastic compression material within the underlay surface thereof for applying the pad on each side of a human nose, within the Maxillary Sinus areas. The Sinus Nasal Pads placed in the Maxillary Sinus area enables the Nasal Passage to open and providing relief from nasal congestion.

### FIELD OF DISCLOSURE

The present disclosure generally relates to sinus pressure relief.

### BACKGROUND

During constant visits to the VA Hospital for Sinus related issues, none of the prescribed medication (Sodium Chloride Nasal Solution, Flunisolide 0.025% Nasal Spray, Cetirizine 10 mg Nasal Tablets) completely assisted my issues. The Nasal Sprays burned or dried out my nasal passages, and the tablets had dizziness side effects. My last line of hope was different branded Nasal strips; less spray meant less burning and dryness, less pills meant less dizziness. The Nasal Strips worked briefly however, due to the shape/structure of my nose, the strip always came off on one side or didn't fully reach both areas as instructed. I researched via Internet for an alternative product like Sinus Pads and was unsuccessful.

### BRIEF OVERVIEW

To solve the aforementioned problem, I carefully cut the strips in a circular shape and placed each on both sides of my Maxillary Sinus passages with an almost 100% success rate. The last concern was ensuring the pads remained for the duration of the night/sleep so I cut the adhesive area of a band aide into a slightly larger circular shape and place the smaller circular strip inside of the circular adhesive area to create a thicker pressure area. By adding the additional adhesive overlay, it created a much stronger, stable adhesive with a center sinus pressure underlay. This is how I created the SINU Pads.

Embodiments of the present disclosure may be referred to as SINU Pads. SINU Pads come in a pair and used to relieve pressure in the Maxillary Sinus area to reduce and/or eliminate snoring, nasal congestion, and sinus headaches associated with sinus pressure. SINU Pads are adhesive type pressure pads placed over the narrow Maxillary Sinus passages on both sides of your face to relieve sinus pressure. SINU Pads were created by Inventor Derrick D. Earls on July 16, 2014.

This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

A two layered device for opening nasal passages, the device comprising: a circular pad having an adhesive underlay surface, a smooth overlay surface, and a center soft plastic compression material within the underlay surface thereof for applying the pad on each side of a human nose, within the Maxillary Sinus areas. The Sinus Nasal Pads placed in the Maxillary Sinus area enables the Nasal Passage to open and providing relief from nasal congestion.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the Applicant. The Applicant retains and reserves all rights in its trademarks and copyrights included herein, and grants permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure. In the drawings:

FIG. 1: is a top perspective view of the invention;

FIG. 2: is a bottom perspective view of the invention;

FIG. 3: is a side perspective view;

FIG. 4: is a front view of the Human Sinus Membrane;

FIG. 5: is a side view of the Human Nose;

FIG. 6: is an exploded side view of the Human Nose;

FIG. 7: is a side view of the Human Nose and invention; and

FIG. 8: is a front view of the Human Face and invention.

### DETAILED DESCRIPTION

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

This invention provides an adhesively-applied nasal sinus pad and methods for substantially preventing a nasal wall tissue of a nose from drawing in during breathing.

As used properly, this invention should assist in the elimination of excessive sinus pressure, snoring, and nasal wall tissue from drawing in during breathing.

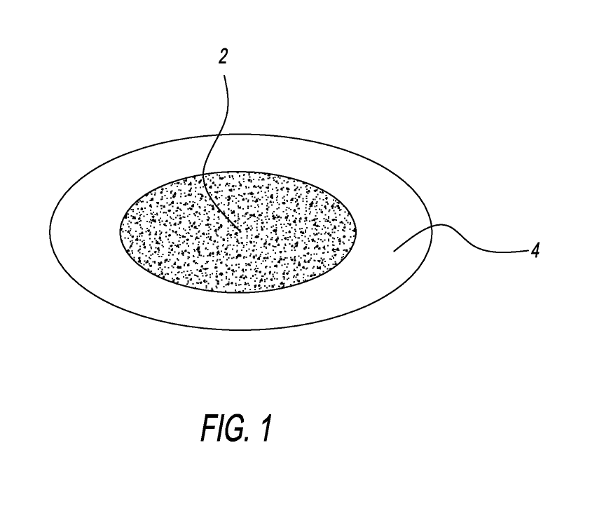

FIG. 1 is a top perspective view of the invention with (2) representing the porous membrane compression center and (4) representing the smooth outer top surface of this invention.

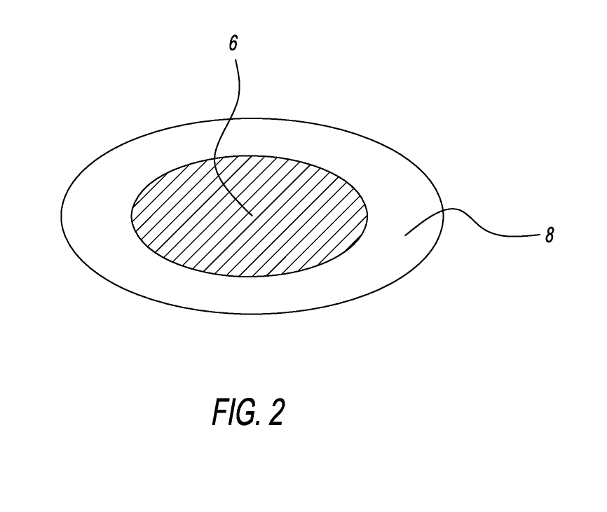

FIG. 2 is a bottom perspective view of the invention with (6) representing the rigid porous membrane compression center and (8) representing the adhesive outer bottom surface, applying adhesive layer to face of this invention.

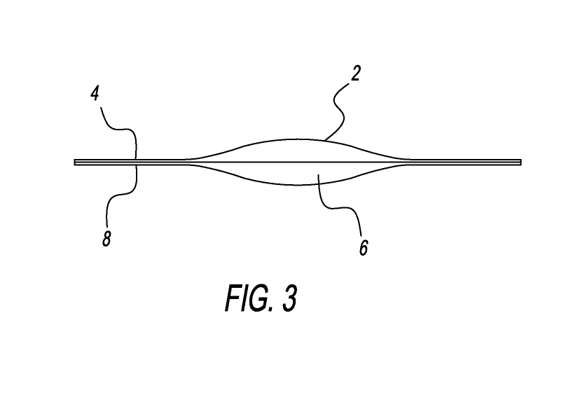

FIG. 3 is a side perspective view, with (4) representing a top perspective side view of the smooth outer top surface of the invention, (8) representing a bottom perspective side view of adhesive outer bottom surface to apply adhesive layer to face, (2) representing a top perspective side view of the porous membrane compression center, and (6) representing a bottom perspective side view of the rigid porous membrane compression center; exploded view of the invention of FIGS. 1 & 2.

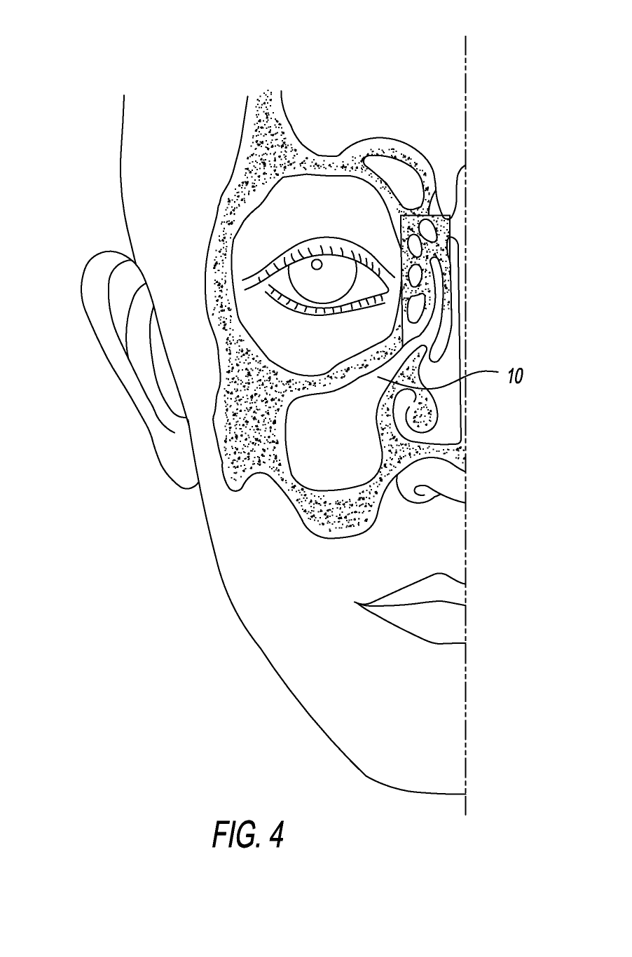

FIG. 4 is a front view of the Human Sinus Membrane with (10) representing the Maxillary Sinus.

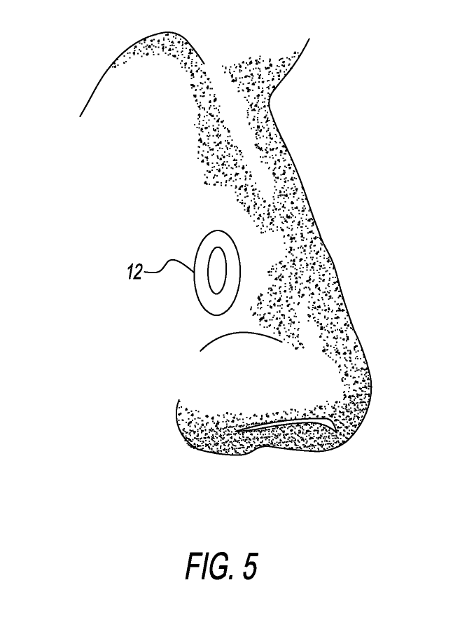

FIG. 5 is a side view of the Human Nose with (12) representing the invention placed in the Maxillary Sinus area.

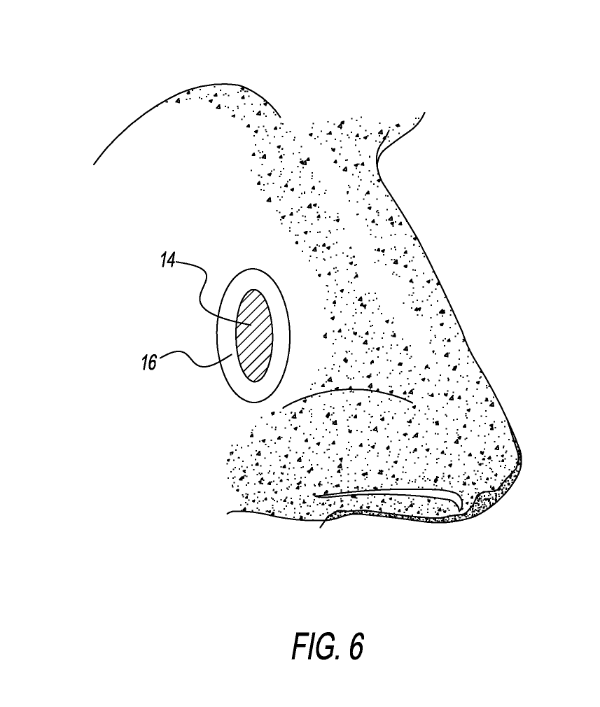

FIG. 6 is an exploded side view of the Human Nose and invention placed on the Maxillary Sinus area with (14) representing the bottom rigid porous membrane compression center and (16) representing the adhesive outer bottom surface.

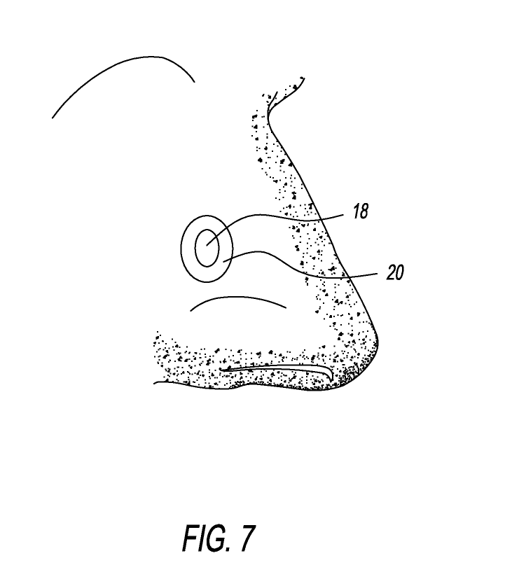

FIG. 7 is a side view of the Human Nose and invention placed on the Maxillary Sinus area with (18) representing the porous membrane compression center and (20) representing the smooth outer top surface of this invention.

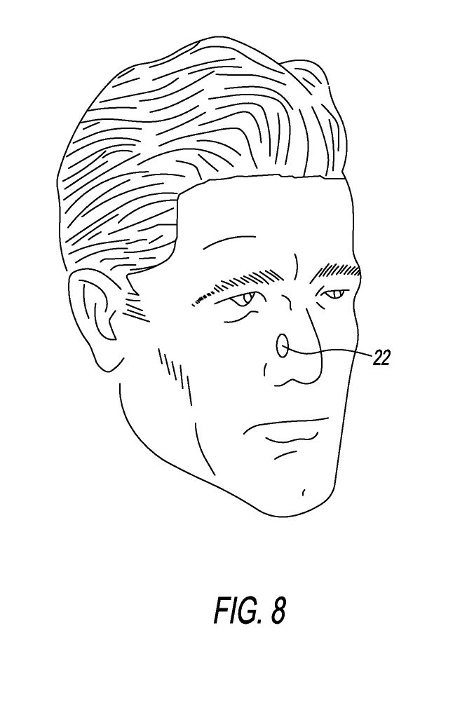

FIG. 8 is a front view of the Human Face and invention placed on the Maxillary Sinus area of the human Face with (22) representing the placement on the sinus facial area of this invention.

Embodiments of the present disclosure may provide a device for opening nasal passages, the device comprising: a circular pad of material having an adhesive on one surface thereof for applying the pad on each side of a human nose; within the Maxillary Sinus areas.

In some embodiments, the pad of material comprises at least two layers and wherein the under laying adhesive area applied to each side of human nose, and the top surface being smooth.

In some embodiments, the center of the underlay pad of material is fabricated from a soft plastic material surrounded by an adhesive backing

In some embodiments, the under laying center pad of material is fabricated from a soft plastic material.

In some embodiments, at least one biasing material is adhered to the surface of the pad of material.

Additional embodiments of the present disclosure may provide a device for opening nasal passages, the device comprising: a two part pad of material having an adhesive underlay surface with a center soft plastic circular compression part within the underlay, a smooth overlay surface, thereof for applying the pads on each side of a human nose.

Yet further embodiments of the present disclosure may provide a method for opening nasal passages, the method comprising: applying an circular pad to each side of the human nose, the pad having a circular shape upon being placed in the Maxillary Sinus area which compresses the nasal passages of the human nose toward an open position; and compressing the nasal passages in an open position.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

1. A device for opening nasal passages, the device comprising: a circular pad of material having an adhesive on one surface thereof for applying the pad on each side of a human nose within the Maxillary Sinus areas.

2. The device of claim 1, wherein the pad of material comprises at least two layers and wherein the under laying adhesive area is configured to be applied to each side of human nose, and the top surface being smooth.

3. The device of claim 2, wherein the center of the underlay pad under laying adhesive area of material is fabricated from a soft plastic material surrounded by an adhesive backing.

4. The device of claim 2, wherein the under laying center pad of material is fabricated from a soft plastic material.

5. The device of claim 1, wherein at least one biasing material is adhered to the surface of the pad of material.

6. A device for opening nasal passages, the device comprising: a two part pad of material having an adhesive underlay surface with a center soft plastic circular compression part within the underlay, a smooth overlay surface, thereof for applying the pads on each side of a human nose.

7. A method for opening nasal passages, the method comprising: applying a circular pad to each side of the human nose, the pad having a circular shape upon being placed in the Maxillary Sinus area which compresses the nasal passages of the human nose toward an open position; and compressing the nasal passages in the open position.
